(19) United States
(12) Patent Application Publication
(10) Pub. No.: US 2008/0038817 A1
(43) Pub. Date: Feb. 14, 2008

(54) Method of harvesting tissue intraoperatively

(75) Inventors: Christopher M. Lyons, Hernando, MS (US); Stanley W. Olson, Germantown, TN (US)

Correspondence Address: LARSON NEWMAN ABEL POLANSKY & WHITE, LLP, 5914 WEST COURTYARD DRIVE, SUITE 200, AUSTIN, TX, 78730, US

(73) Assignee: WARSAW ORTHOPEDIC, INC., Warsaw, IN (US)

(21) Appl. No.: 11/501,376

(22) Filed: Aug. 9, 2006

(51) Int. Cl. C12N 5/08

(52) U.S. Cl. 435/325; 435/366

(57) ABSTRACT

A method of harvesting tissue intraoperatively is disclosed and can include exposing a joint in a patient. Further, the method can include resecting tissue from the joint and identifying healthy tissue. The method can also include placing the healthy resected tissue in a container with a tissue preservation fluid.

### FIELD OF THE DISCLOSURE

The present disclosure relates generally to orthopedics and orthopedic surgery. More specifically, the present disclosure relates to harvesting tissue during orthopedic surgeries.

### BACKGROUND

Numerous joint replacement surgeries are performed on patients each year. For example, knee joints can be replaced, hip joints can be replaced, shoulder joints can be replaced, etc. Typically, a joint is replaced because tissue within the joint is diseased and is causing the patient pain. For example, a portion of a femur, e.g., a condyle, can be arthritic and can be causing inflammation and joint pain.

During a joint replacement surgery, bone tissue and other tissue, e.g., meniscal tissue, can be resected from the adjoining bones that make up the joint. In many cases, only a portion of the joint is diseased, i.e., in a knee replacement only a lateral condyle of the femur or only a medial condyle of the femur may be diseased. The resected tissue is typically discarded. However, some of the resected tissue may be healthy, but may be removed anyway in order to allow the artificial joint to be properly positioned.

Accordingly, there is a need for a method of harvesting tissue intraoperatively.

### BRIEF DESCRIPTION OF THE DRAWINGS

FIG. 1 is a plan view of a femur;

FIG. 2 is a plan view of a tibia and fibula;

FIG. 3 is a plan view of a knee joint;

FIG. 4 is a flow chart of a method of harvesting tissue intraoperatively; and

FIG. 5 is a general diagram representing a tissue bank.

### DETAILED DESCRIPTION OF THE DRAWINGS

A method of harvesting tissue intraoperatively is disclosed and can include exposing a joint in a patient. Further, the method can include resecting tissue from the joint and identifying healthy tissue. The method can also include placing the healthy resected tissue in a container with a tissue preservation fluid.

In another embodiment, a tissue specimen is disclosed and can include healthy tissue harvested during an orthopedic surgery and at least one identifying characteristic associated with the healthy tissue.

In still another embodiment, a tissue bank is disclosed and can include a first tissue specimen, a second tissue specimen, a third tissue specimen, and an Nth tissue specimen. Each tissue specimen can be harvested intraoperatively, placed in a container having a tissue preservation fluid therein, and transferred to the tissue bank.

### Description of Relevant Anatomy

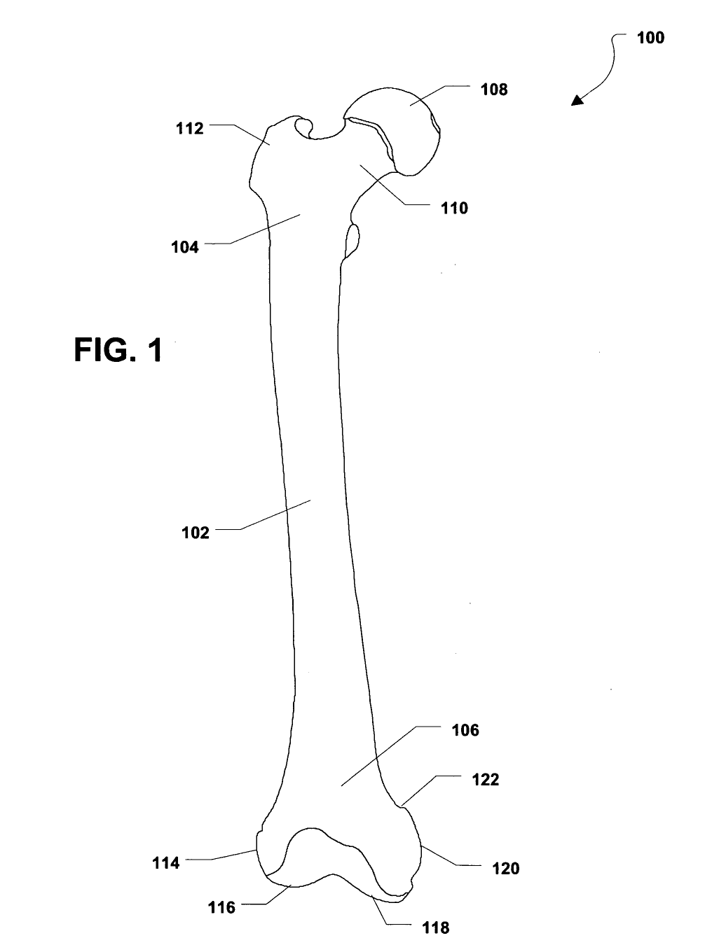

Referring to FIG. 1, a femur is shown and is generally designated 100. As shown, the femur 100 includes a femoral body 102 that can define a proximal end 104 and a distal end 106. Further, the femur 100 can include a femoral head 108 that extends from the proximal end 104 of the femoral body 102. Further, a neck 110 can be established between the femoral head 108 and the femoral body 102. In a particular embodiment, the femoral head 108 can fit into a hip socket, a.k.a., an acetabulum (not shown).

As further illustrated in FIG. 1, the proximal end 104 of the femoral body 102 can include a greater trochanter 112 adjacent to the neck of the proximal end 104. Additionally, the distal end 106 of the femoral body 102 can include a lateral epicondyle 114, a lateral condyle 116, a medial condyle 118, and a medial epicondyle 120. In a particular embodiment, the lateral condyle 116 and the medial condyle 118 can articulate with a patella (not shown). FIG. 1 also indicates that the femur 100 can include an adductor tubercle 122.

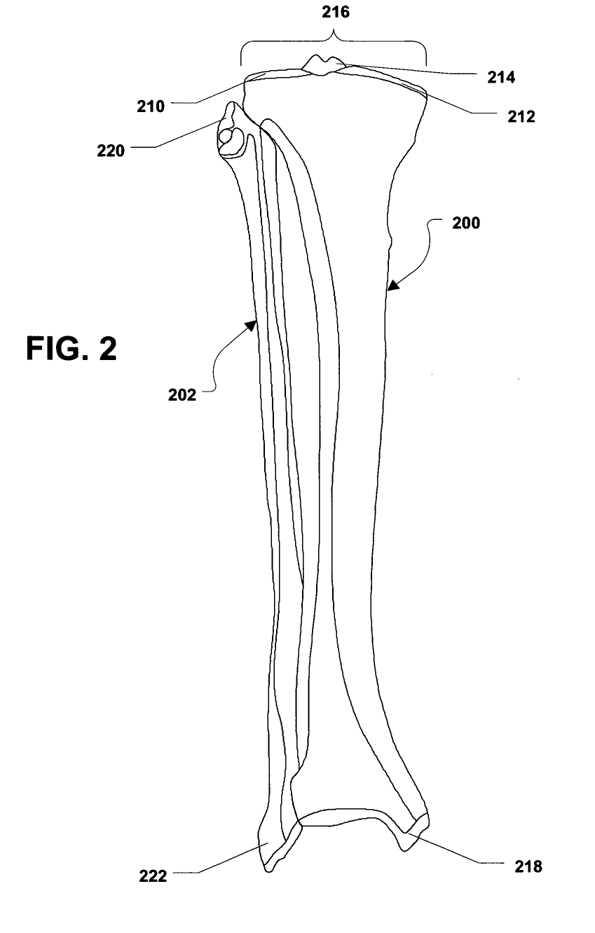

Referring to FIG. 2, a tibia is shown and is generally designated 200. Further, a fibula is shown and is generally designated 202. As shown in FIG. 2, the tibia 200 can include a lateral condyle 210 and a medial condyle 212. Further, the tibia 200 can include a tibial spine 214 that extends from the tibia 200 between the lateral condyle 210 and the medial condyle 212. The lateral condyle 210, the medial condyle 212, and the tibial spine 214 can establish an articular capsule 216. Further, as shown, the tibia 200 can include a medial malleolus 218. As illustrated in FIG. 2, the fibula 202 can include a styloid process 220 and a lateral malleolus 222.

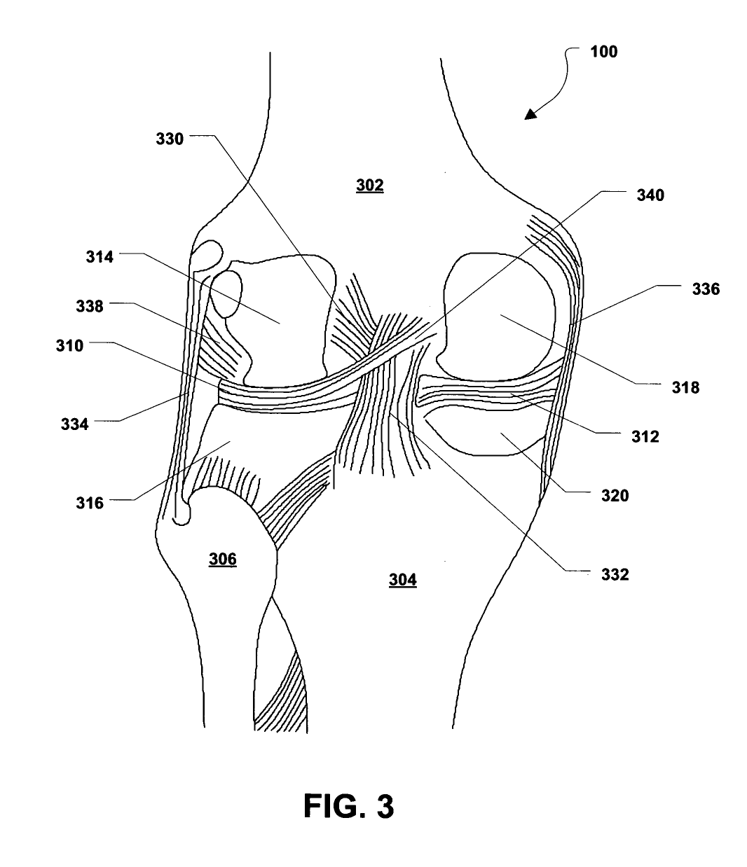

FIG.3 illustrates a knee joint that is generally designated 300. In general, the knee joint 300 can be established at the interface between a femur 302, a tibia 304, and a fibula 306. In particular, the knee joint 300 can include a lateral meniscus 310 and a medial meniscus 312. The lateral meniscus 310 can be sandwiched between a lateral condyle 314 of the femur 302 and a lateral condyle 316 of the tibia 304. Further, the medial meniscus 312 can be sandwiched between a medial condyle 318 of the femur 302 and a medial condyle 320 of the tibia 304.

As depicted in FIG.3, the knee joint 300 can further include an anterior cruciate ligament 330, a posterior cruciate ligament 332, fibular collateral ligament 334, and a tibial collateral ligament 336. The knee joint 300 can also include a Popliteus tendon 338 and a Wrisberg ligament 340.

During a full knee replacement surgery, portions of the knee joint 300 may be resected. For example, the lateral meniscus 310 and the medial meniscus 312 may be resected. Further, the lateral condyle 314 and the medial condyle 318 of the femur 302 may be resected in order to shape the end of the femur 302 to receive a superior portion of an artificial knee joint. Also, the lateral condyle 316 and the medial condyle 320 of the tibia 304 may be resected in order to shape the end of the tibia 304 to receive an inferior portion of an artificial knee joint.

Commonly, only a portion of the knee joint 300 is diseased, e.g., arthritic. As such, the lateral meniscus 310 or the medial meniscus 312 may be the only joint component that is diseased. However, during the full knee replacement surgery the lateral meniscus 310 and the medial meniscus 312 are typically resected even if one or the other is healthy tissue. Further or alternatively, the lateral condyle 314 or the medial condyle 318 of the femur 302 may be the only joint component that is diseased. Typically, however, the lateral condyle 314 and the medial condyle 318 of the femur 302 are both removed during full knee replacement surgery, regardless of whether one or the other is healthy tissue. Further or alternatively, the lateral condyle 316 or the medial condyle 320 of the tibia 304 may be the only joint component that is diseased. However, in most cases the lateral condyle 316 and the medial condyle 320 of the tibia 304 are both removed during full knee replacement surgery, regardless of whether one or the other is healthy tissue.

### Description of a Method of Harvesting Tissue Intraoperatively

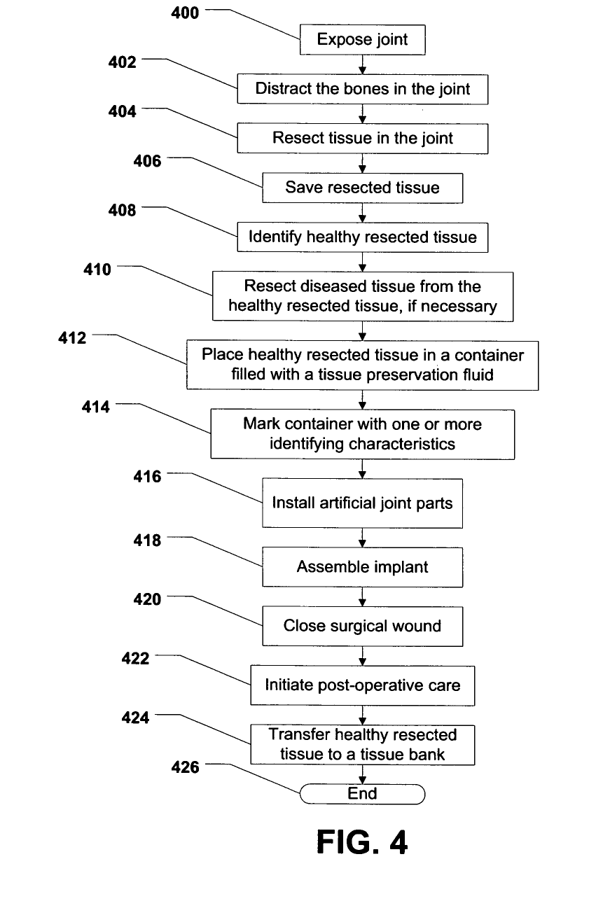

Referring to FIG. 4, various steps of an exemplary method of harvesting tissue intraoperatively are shown. In a particular embodiment, the method of harvesting tissue can be performed during a knee replacement surgery. However, the method of harvesting tissue can be performed during a hip replacement surgery, a shoulder replacement surgery, or during another joint replacement surgery.

Commencing at block 400, a surgeon can expose a joint. At block 402, the surgeon can distract the bones that make up the joint. For example, if the joint is a knee joint, the surgeon can distract the femur from the tibia and fibula. Moving to block 404, the surgeon can resect tissue in the joint in order to prepare the joint to receive the components of the artificial joint to be installed during the joint replacement surgery. For example, during a knee replacement surgery, the surgeon can resect a lateral meniscus, a medial meniscus, a lateral condyle of the femur, a medial condyle of the femur, a lateral condyle of the tibia, a medial condyle of the tibia, or a combination thereof. In a particular embodiment, when the lateral condyle and the medial condyle of the tibia are resected, they can be resected as a single piece of tissue known as the tibial plateau.

Proceeding to block 406, the resected tissue can be saved (i.e., not discarded) by the surgeon. At block 408, the healthy resected tissue can be identified. Further, at block 410, diseased tissue can be resected from the healthy tissue, if necessary. For example, when a tibial plateau is resected, either the lateral condyle or the medial condyle of the tibia may be diseased and the remaining condyle may be healthy. As such, the diseased tissue can be resected from the healthy tissue.

Continuing to block 412, the healthy resected tissue can be placed in a container filled with tissue preservation fluid. The tissue preservation fluid can increase the longevity of the tissue while it is stored for later use. Further, the tissue preservation fluid can provide for chondropreservation of cartilage, ligaments, tendons, etc. Moreover, the tissue preservation fluid can condition, or otherwise optimize, the tissue placed therein by supplying nutrients, one or more growth factors, or a combination thereof, to fortify the tissue for reimplantation. As such, the tissue preservation fluid may not only preserve the tissue, but the tissue preservation fluid can increase the vitality or fitness of the tissue such that the tissue may be more durable and more readily incorporated into a patient in which the tissue is implanted.

At block 414, the container can be marked with one or more identifying characteristics. For example, the container can be marked with a location identifier, i.e., the location from which the tissue was harvested. The container can also be marked with a size identifier, i.e., the thickness of the tissue and other relevant dimensions. Further, the container can be marked with an amount identifier, e.g., the amount of resected tissue. The container can also be marked with a type identifier, e.g., the type of tissue resected. Moreover, the container can be marked with a blood identifier, e.g., a blood type associated with the patient from whom the tissue was harvested. Additionally, the container can be marked with a date identifier and a time identifier indicating the time and date at which the tissue was harvested. In like manner, the container can be marked with any other indicia of potentially useful information, such as the age or sex of the patient from whom the tissue was harvested or the like.

Moving to block 416, the parts of the artificial joint can be installed on the bones that make up the joint. At block 418, the implant can be assembled. Further, at block 420, the surgical wound can be closed. The surgical wound can be closed using sutures, surgical staples, or any other surgical technique well known in the art. Moving to block 422, postoperative care can be initiated. At block 424, the healthy resected tissue can be transferred to a tissue bank. The method can end at step 426.

### Description of a Tissue Bank

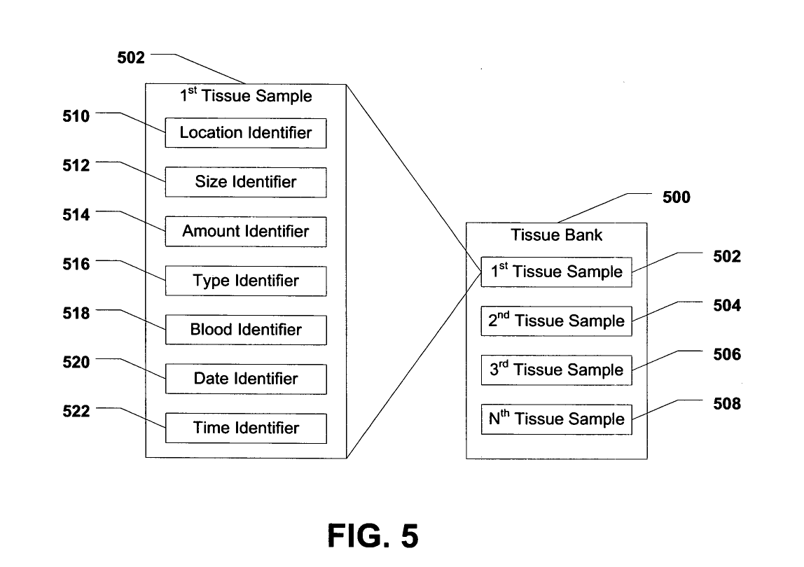

Referring to FIG. 5, a tissue bank is shown and is generally designated 500. As shown, the tissue bank 500 can include a first tissue specimen 502, a second tissue specimen 504, a third tissue specimen 506, and an Nth tissue specimen 508. In a particular embodiment, each tissue specimen 502, 504, 506, 508 can be harvested using the method described above.

As shown, the first tissue specimen 502 can include a location identifier 510 that can indicate the location from which the first tissue specimen 502 was harvested. For example, the location can be a knee joint, a shoulder joint, a hip joint, or some other joint. The first tissue specimen 502 can also include a size identifier 512 that can indicate the thickness of the first tissue specimen 502 or some other relevant dimensions. Further, the first tissue specimen 502 can include an amount identifier 514 that can indicate an amount of tissue included in the first tissue specimen 502. The first tissue specimen 502 can also include a type identifier 516 that can indicate the type of tissue resected. Moreover, as shown in FIG. 5, the first tissue specimen 502 can include a blood identifier 518 that can indicate a blood type associated with the patient from which the first tissue specimen 502 was harvested. Additionally, the first tissue specimen 502 can include a date identifier 520 and a time identifier 522. The date identifier 520 and the time identifier 522 can indicate the date and time at which the first tissue specimen 502 was harvested. In like manner, the first tissue specimen can include any other indicia of potentially useful information, such as the age or sex of the patient from whom the tissue was harvested or the like.

### CONCLUSION

With the configuration of structure described above, the method of harvesting tissue intraoperatively provides a way to capture tissue that would otherwise be discarded. The healthy tissue harvested using the method described herein can be transferred to a tissue bank and saved for later use. Further, the healthy tissue can be used for partial tissue transplants in patients. As such, patients that would otherwise have to undergo a more invasive full joint replacement have a less invasive option that may heal relatively quicker than a full joint replacement. The tissue can be kept at a tissue bank within the hospital in which the tissue is harvested or a central location that serves multiple hospitals and surgical centers.

The above-disclosed subject matter is to be considered illustrative, and not restrictive, and the appended claims are intended to cover all such modifications, enhancements, and other embodiments that fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

1. A method of harvesting tissue intraoperatively, the method comprising:
exposing a joint in a patient;
resecting tissue from the joint;
identifying healthy tissue; and
placing the healthy resected tissue in a container with a tissue preservation fluid.

2. The method of claim 1, further comprising resecting diseased tissue from the healthy tissue.

3. The method of claim 1, further comprising marking the container with at least one identifying characteristic.

4. The method of claim 3, further comprising transferring the healthy tissue to a tissue bank.

5. The method of claim 3, wherein the at least one identifying characteristic includes a location identifier, a size identifier, an amount identifier, a type identifier, a blood identifier, a date identifier, a time identifier, or a combination thereof.

6. The method of claim 5, wherein the location identifier indicates a type of joint from which the healthy tissue is harvested.

7. The method of claim 5, wherein the size identifier indicates a dimension of the healthy tissue.

8. The method of claim 5, wherein the amount identifier indicates an amount of the healthy tissue.

9. The method of claim 5, wherein the type identifier indicates a tissue type associated with the healthy tissue.

10. The method of claim 5, wherein the blood identifier indicates a blood type associated with the patient from which the healthy tissue is harvested.

11. The method of claim 5, wherein the date identifier indicates a date on which the healthy tissue is harvested.

12. The method of claim 5, wherein the time identifier indicates a time at which the healthy tissue is harvested.

13. A tissue specimen, comprising:
healthy tissue harvested during an orthopedic surgery; and
at least one identifying characteristic associated with the healthy tissue.

14. The tissue specimen of claim 13, wherein the healthy tissue is harvested during a joint replacement surgery.

15. The tissue specimen of claim 14, wherein the healthy tissue is harvested during a knee replacement surgery, a hip replacement surgery, or a shoulder replacement surgery.

16. The tissue specimen of claim 13, further comprising:
a container; and
a tissue preservation fluid, wherein the tissue specimen and the tissue preservation fluid are placed within the container.

17. The tissue specimen of claim 13, wherein the at least one identifying characteristic includes a location identifier, a size identifier, an amount identifier, a type identifier, a blood identifier, a date identifier, a time identifier, or a combination thereof.

18.-24. (canceled)

19. A tissue bank, comprising:
a first tissue specimen;
a second tissue specimen;
a third tissue specimen; and
an Nth tissue specimen, wherein each tissue specimen is harvested intraoperatively, placed in a container having a tissue preservation fluid therein, and transferred to the tissue bank.

20. The tissue bank of claim 19, wherein each tissue specimen comprises:
healthy tissue harvested during an orthopedic surgery; and
at least one identifying characteristic associated with the healthy tissue.

21. The tissue bank of claim 20, wherein the healthy tissue is harvested during a joint replacement surgery.

22. The tissue bank of claim 20 wherein the healthy tissue is harvested during a knee replacement surgery, a hip replacement surgery, or a shoulder replacement surgery.

23. The tissue bank of claim 20, wherein the at least one identifying characteristic includes a location identifier, a size identifier, an amount identifier, a type identifier, a blood identifier, a date identifier, a time identifier, or a combination thereof.

24.-36. (canceled)
